(19) United States
(12) Patent Application Publication
(10) Pub. No.: US 2009/0012605 A1
(43) Pub. Date: Jan. 8, 2009

(54) Coronary stent that releases medicamentuous composition to prevent and treat restenosis and fabrication process

(76) Inventors: Alexander Do Canto Zago, Porto Alegre (BR); Alcides Jose Zago, Porto Alegre (BR)

Correspondence Address: PATENT LAW AGENCY, LLC, 3146 NORTH VERDUGO ROAD, GLENDALE, CA, 91208-1665, US

(21) Appl. No.: 12/215,936

(22) Filed: Jul. 1, 2008

Related U.S. Application Data
(63) Continuation of application No. PCT/BR2007/000004, filed on Jan. 3, 2007.

(30) Foreign Application Priority Data
Jan. 3, 2006 (BR) PI 0600275-7

(51) Int. Cl. A61F 2/06

(52) U.S. Cl. 623/1.42

(57) ABSTRACT

A stent is described that releases medicamentuous composition to prevent and treat restenosis and the fabrication process that comprehends between 10.0 to 500.0 Ug/cm² of the surface of the stent coated with rapamicyn (sirolismus) or analogous and between 0.01 to 20.0 Ug/mm² of the surface of the stent coated with paclitaxel or analogous.

### CROSS-REFERENCE TO RELATED APPLICATIONS

This Application is a CONTINUATION of a co-pending International Patent Application No. PCT/BR2007/000004 with an international filing date of 3 Jan. 2007 that designated the United States of America, which claims the benefit of priority of the Federative Republic of Brazil Patent Application No. PI 0600275-7, with a filing date of 3 Jan. 2006, the entire disclosures of all Applications is expressly incorporated by reference in their entirety herein.

### BACKGROUND OF THE INVENTION

1. Field of the Invention

The present invention regards to a stent that releases medicamentuous composition to prevent and treat restenosis and fabrication process. Specifically includes a stent containing a therapeutic composition with two cellular antiproliferative drugs that act in different phases of the cellular cycle.

2. Description of Related Art

Half of the accounted deaths in the world occur due coronary diseases, according to health authorities. Due to the importance of the problem, scientists worldwide dedicate a considerable time of their work to the formulation of more efficient drugs and methods to fight these infirmities.

The introduction of the coronary stent implant is the second greatest advance in the treatment of obstructive percutaneous coronarypathy since the introduction of angioplasty with the balloon. The stent implant prevents almost completely the abrupt obstruction of the blood vessels and has reduced significantly the late restenosis by the elimination of the acute elastic retraction of the blood vessel and, particularly by the chronic negative remodeling of the blood vessel.

The first stents were rudimentary, creating problems in the correct implantation and high taxes of subacute thrombosis. Since then, the stent technology with stents has significatively improved, resulting in more flexible stents, very low profile and, consequently, easier to be placed. Currently the stents implantation is a safe and predictable technique. It has reduced significantly the need of rescue emergency surgery, allowing the percutaneous treatment of a wide variety of obstructive coronary injuries.

Nonetheless, it might occur neointimal hyperplasia in the interior of the stent, either being focal or diffuse, generating clinically significant obstructions in 15% a 20% of the cases. The obstruction after the implant occurs by the creation of an exaggerated scar through the structure of the stent. This metal frame provokes a serious trauma in the coronary tissue and a strong immunological reaction in the organism of some patients. The result is the narrowing of the blood vessel.

A way to handle the occurrence of thrombosis in the stent and late restenosis in the stent could be due an increase of the blood compatibility and tissue of the stent by coating of the stent, which might be passive or active. The passive coatings, such as the polymers or inorganic ones provide a inert biologically barrier between the surface of the stent, the wall of the blood vessel and circulatory blood, in an attempt to decrease the anti-inflammatory responses and prevent thrombosis in the stent and neointimal hyperplasia. The active coatings are biologically active because they are carried with drugs (heparin, paclitaxel or rapamicyn) that are released, at a certain dose, to prevent the occurrence of thrombosis or restenosis.

The rapamicyn or sirolimus is a potent agent antiproliferative that acts at the phases G1-S of the cellular cycle. It also has antibiotic, antifungal and immunosuppressor activities. As a cellular antiproliferative agent it has being used in coronary stents, providing significant reduction in the taxes of hyper proliferation of neointimal intra-stent, that is, the re-obstruction of the coronary artery after stent implant by the unordered and excessive proliferation of endothelial and muscular flat cells in the interior of the stent.

The paclitaxel is an antiproliferative cellular agent that acts at the end of the cellular cycle at the phase G2-M. Developed as an antineoplastic drug, it is presently also used in coronary stents due to the efficacy in the reduction of the number of intra-stent restenosis.

The patent WO 03/037397 describes a composition embedded in a stent that comprehends, at least, a bioabsorbable polymer such as polyester and a therapeutic substance such as sirolimus, actinomycin-d and paclitaxel.

The patent US2004/02432226 describes a stent embedded with a drug with antirestenoic characteristics selected from a group that includes alkeran, cytoxan, leukeran, cis-platinun, bicnu, adriamycin, doxorubicin, cerubidine, idamycin, mithracin, mutamycin, fluorouracil, methotrexate, thoguanine, toxotere, etoposide, vincristine, irinotecan, hycampatin, matulane, vumon, hexalin, hydroxyurea, gemzar, oncovin, etophophos, tacrolimus (fk506) and the following analogous of rapamicyn: sdz-rad, cci-7790, 7-epi.rapamicyn, 7-epi-thiomethyl-rapamicyn, 7-epi-trimethoxyphenyl-rapamicyn, 7-epi-thiomethyl-rapamicyn, 7-demethoxy-rapamicyn, 32-demethoxy, 2-desmethyl and proline.

The patent WO 2004/110302 describes a method to decrease the level of restenosis through the application of a stent with continuous administration of a dose of an antirestenoic agent, such as the paclitaxel.

The patent CA 2,269,310 describes a method to deliver rapamicyn locally, in a stent-body or mixed or limited to a coating of a polymer applied in a stent to prevent restenosis.

The patent US 2003/0176915 describes a stent that holds rapamicyn mixed or limited to a coating of polymer to prevent the restenosis.

The patent US 2005/0085902 describes a method of treatment of the cardiovascular disease by the implementation of a stent with the release of rapamicyn.

Therefore the use of stents embedded in drugs constitutes the most significant advance in the field of interventionist cardiology. The technical literature present stents embedded with rapamicyn and analogous or paclitaxel and analogous that provoke the decrease of the number of restenosis of 25-30% from the stents non-pharmacological to 7-8% in the pharmacological stents.

Nonetheless, the cellular antiproliferative effect of the rapamicyn and analogous and the paclitaxel and analogous through the performance in different phases of the cellular cycle suggest a synergetic effect of both. Therefore, the lack of success of one of the drugs in the blockage of one or more metabolic routes of the cellular proliferation is balanced by the success of the other drug in the blockage of this or more routes.

Consequently, the technical literature show coronary stents releasing drugs that although solve the problem of intra-stent neointimal hyper proliferation partially, still there is the need to improve the medicamentuous composition with the incorporation of two synergetic action antiproliferative cellular drugs, meaning to act in different phases of the cellular cycle.

Therefore, the open literature do not describes nor suggests a stent that releases medicamentuous compositions to prevent and treat the restenosis that have two cellular antiproliferative drugs of synergetic action, such stent that is being described and being claimed in the present application.

### BRIEF SUMMARY OF THE INVENTION

Generally, the present invention concerns to a stent that releases medicamentuous composition to prevent and treat restenosis that comprehends 10.0 to 500.0 Ug/cm2 of the surface of the stent coated with rapamicyn (sirolimus) or analogous and between 0.01 to 20.0 Ug/mm2 of the surface of the stent coated with paclitaxel or analogous. Still, the present invention refers to a fabrication process of a stent that releases medicamentuous composition.

One of the materialization of this invention comprehends a stent embedded with two cellular antiproliferative drugs that act in different phases of the cellular cycle.

These and other features, aspects, and advantages of the invention will be apparent to those skilled in the art from the following detailed description of preferred non-limiting exemplary embodiments, taken together with the claims that follow.

### BRIEF DESCRIPTION OF THE DRAWINGS

None

### DETAILED DESCRIPTION OF THE INVENTION

The detailed description set forth below is intended as a description of presently preferred embodiments of the invention and is not intended to represent the only forms in which the present invention may be constructed and or utilized.

The coronary stent that releases medicamentuous composition to prevent and treat restenosis, object of the present invention, comprehends between 10.0 to 500.0 Ug/cm2 of the surface of the stent coated with rapamicyn (sirolimus) or analogous and between 0.01 to 20.0 Ug/mm2 of the surface of the stent coated with paclitaxel or analogous. Preferably, the stent is coated with rapamicyn or analogous in intervals between 80.0 to 240.0 Ug/cm2 of the surface and with paclitaxel in intervals between 0.1 to 10.0 Ug/mm2 of the surface.

All of the proposed compositions by the invention have rapamicyn or analogous that includes biolimus, everolimus or zotarolimus, and paclitaxel and analogous, that includes the docetaxel.

The process of impregnation of the drugs rapamicyn and analogous and raclitaxel and analogous is made in four modalities.

Modality 1:

To mix the rapamicyn or analogous with the paclitaxel or analogous in a proportion that varies from 20 to 80% and with one or more polymers, through the solubilization in adequated organic solvent; with further application of the mixture of embedded polymers in the rapamicyn active components or analogous and paclitaxel or analogous on the surface of the stent.

Modality 2:

Embed one or more polymers with the rapamicyn or analogous and with paclitaxel or analogous. Mix the polymers embedded with the active components in a proportion that varies from 20 to 80%. Apply the mixture of polymers embedded in the active components on the surface of the stent.

Modality 3:

Mix the rapamicyn or analogous with the paclitaxel or analogous in a proportion that varies from 20 to 80%. Embed one or more polymers in the mixture of the active components and apply the polymer mixture embedded in the active components on the surface of the stent.

Modality 4:

Embed one or more polymers with the rapamicyn or analogous and one or more polymers with paclitaxel or analogous. Apply the polymers embedded with the rapamicyn or analogous and the polymers embedded with paclitaxel or analogous in alternate layers on the surface of the stent.

Tests done in swine attest the efficacy of the combination of rapamicyn and paclitaxel in the coating of stents to prevent the restenosis.

Twelve coronary stents, commercially available, and sizing 3.0×18 mm were used in the study: (a) three were coated with bioabsorbable polymer and rapamicyn, (b) three were coated with bioabsorbable polymer and paclitaxel, (c) three were coated with bioabsorbable polymer, rapamicyn and paclitaxel and (d) three were not coated. Six swine with the descendent front left artery with a diameter of approximately 2.75 mm were submitted to the implant of the stent above mentioned through fluoroscopy. Three of these swine were submitted to the implantation of three stents not coated in the transition of the proximal to medium third of the descendent front left artery and three stents coated with bioabsorbable polymer, rapamicyn and paclitaxel in the medium third of the same coronary artery. The other three swine were submitted to the implant of three stents coated with bioabsorbable polymer and paclitaxel in the transition of the proximal to medium third of the descendent artery left artery and of three stents coated with bioabsorbable polymers and rapamicyn in the medium third of the same coronary artery. At the end of the procedure a control angiography was made and intravascular ultrasound to analyze the expansion and apposition of the stent, as well as to evaluate the minimum luminal diameter and minimum luminal area.

In 90 days the swine were re-studied by coronary angiography and with intravascular ultrasound to evaluate the intra-stent restenosis and neointimal proliferation. The calculation of the results of the late loss (LL) and the stenosis diameter (SD) for each stent, following the calculation of the average of these results for each group of stents according to chart 1.

CHART 1
Results of the late loss and stenosis diameter in coronary stents

| | | Stenosis |
|---|---|---|
| | | Diameter |
| Coronary Stent | Late Loss (LL) | (SD) |
| (a) coated with bioabsorbable polymer and | 0.23 mm | 19.5% |
| rapamicyn | | |
| (b) coated with bioabsorbable polymer and | 0.42 mm | 30.5% |
| paclitaxel | | |
| (c) coated with bioabsorbable polymer, | 0.12 mm | 10.3% |
| rapamicyn and paclitaxel | | |
| (d) not coated | 1.48 mm | 58% |

The results obtained make clear the superiority of the coronary stents coated with polymer and drugs related to the non-coated stents, which is known. Nonetheless, also make clear that better results of the drug combination—rapamicyn and paclitaxel, related to the isolated use of rapamicyn or the paclitaxel coating the stents, constitutes a great potential advance in the prevention of restenosis and consequently in the treatment of the coronary arterial disease with stents.

Although the invention has been described in considerable detail in language specific to structural features and or method acts, it is to be understood that the invention defined in the appended claims is not necessarily limited to the specific features or acts described. Rather, the specific features and acts are disclosed as preferred forms of implementing the claimed invention. Stated otherwise, it is to be understood that the phraseology and terminology employed herein, as well as the abstract, are for the purpose of description and should not be regarded as limiting.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, proximal, distal, parallel, perpendicular, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) is not used to show a serial or numerical limitation but instead is used to distinguish or identify the various members of the group.

In addition, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of,” “act of,” “operation of,” or “operational act of” in the claims herein is not intended to invoke the provisions of 35 U.S.C. 112, Paragraph 6.

What is claimed is:

1. Coronary stent that releases medicamentuous composition to prevent and treat restenosis and fabricaton and fabrication process characterized by comprehending between 10.0 and 500.0 Ug/cm2 of the surface of the stent coated with rapamicyn (sirolimus) or analogous and between 0.01 to 20.0 Ug/mm2 of the surface of the coronary stent coated with paclitaxel or analogous.

2. Coronary stent that releases medicamentuous composition to prevent and treat restenosis and fabricaton and fabrication process, according to claim 1, characterized by comprehending between 80.0 to 240.0 Ug/cm2 of the surface of the stent coated with rapamicyn or analogous and between 0.1 to 10.0 Ug/mm2 of the surface coated with paclitaxel or analogous.

3. Coronary stent that releases medicamentuous composition to prevent and treat restenosis and fabricaton and fabrication process, according to claim 1, characterized by the fact to have biolimus, everolimus or zotarolimus.

4. Coronary stent that releases medicamentuous composition to prevent and treat restenosis and fabricaton and fabrication process, according to claim 1, characterized by comprehending docetaxel.

5. Coronary stent that releases medicamentuous composition to prevent and treat restenosis and fabricaton and fabrication process, characterized by comprehending steps of:
To mix the rapamicyn or analogous with the paclitaxel or analogous in a proportion that ranges from 20 to 80% and with one or more polymers, according to the solubilization an adequate organic solvent;
Apply the mixture of polymers embedded in the active components rapamicyn or analogous and paclitaxel or analogous on the surface of the stent.

6. Coronary stent that releases medicamentuous composition to prevent and treat restenosis and fabricaton and fabrication process, characterized by comprehending steps of:
to embed one or more polymers with the rapamicyn or analogous and with paclitaxel or analogous:
to mix the polymers embedded with the active components in a proportion that ranges from 20 to 80%;
to apply the mixture of embedded active components on the surface of the stent.

7. Coronary stent that releases medicamentuous composition to prevent and treat restenosis and fabricaton and fabrication process, characterized by comprehending steps of:
to mix the rapamicyn or analogous with the paclitaxel or analogous in a proportion that ranges from 20 to 80%;
to embed one or more polymers in the mixture of the active components and apply the mixture of the polymers embedded in the active components on the surface of the stent.

8. Coronary stent that releases medicamentuous composition to prevent and treat restenosis and fabricaton and fabrication process, characterized by comprehending steps of:
to embed one or more polymers with the rapamicyn or analogous and one or more polymers with paclitaxel or analogous;
to apply the polymers embedded with rapamicyn or analogous and the polymers embedded with paclitaxel or analogous in alternate layers on the surface of the stent.
